(19) United States
(12) Patent Application Publication
(10) Pub. No.: US 2015/0106029 A1
(43) Pub. Date: Apr. 16, 2015

(54) METHOD OF CHARACTERIZING CRUDE OIL BY HIGH PRESSURE LIQUID CHROMATOGRAPHY

(71) Applicants: Omer Refa KOSEOGLU, Dhahran (SA); Adran AL-HAJJI, Damamam (SA); Saroj Kumar PANDA, Dhahran (SA)

(72) Inventors: Omer Refa KOSEOGLU, Dhahran (SA); Adran AL-HAJJI, Damamam (SA); Saroj Kumar PANDA, Dhahran (SA)

(21) Appl. No.: 13/468,105

(22) Filed: May 10, 2012

Related U.S. Application Data
(60) Provisional application No. 61/501,962, filed on Jun. 28, 2011.

(51) Int. Cl. G06F 19/00; G01N 30/86; G01N 33/28

(52) U.S. Cl. CPC G01N 30/86; G01N 33/2811
USPC 702/23; 702/25

(57) ABSTRACT

A system and a method are provided for calculating the cetane number, octane number, pour point, cloud point and aniline point of a crude oil fractions from the density and high pressure liquid chromatography (HPLC) of a sample of the crude oil.

### RELATED APPLICATION

This application claims priority to U.S. Provisional Patent Application No. 61/501,962 filed on Jun. 28, 2011, which is incorporated herein by reference.

### FIELD OF THE INVENTION

This invention relates to a method and process for the evaluation of samples of crude oil and its fractions by high pressure liquid chromatography (HPLC), avoiding the need to conduct crude oil assays.

### BACKGROUND OF THE INVENTION

Crude oil originates from the decomposition and transformation of aquatic, mainly marine, living organisms and/or land plants that became buried under successive layers of mud and silt some 15-500 million years ago. They are essentially very complex mixtures of many thousands of different hydrocarbons. Depending on the source, the oil predominantly contains various proportions of straight and branched-chain paraffins, cycloparaffins, and naphthenic, aromatic, and polynuclear aromatic hydrocarbons. These hydrocarbons can be gaseous, liquid, or solid under normal conditions of temperature and pressure, depending on the number and arrangement of carbon atoms in the molecules.

Crude oils vary widely in their physical and chemical properties from one geographical region to another and from field to field. Crude oils are usually classified into three groups according to the nature of the hydrocarbons they contain: paraffinic, naphthenic, asphaltic, and their mixtures. The differences are due to the different proportions of the various molecular types and sizes. One crude oil can contain mostly paraffins, another mostly naphthenes. Whether paraffinic or naphthenic, one can contain a large quantity of lighter hydrocarbons and be mobile or contain dissolved gases; another can consist mainly of heavier hydrocarbons and be highly viscous, with little or no dissolved gas. Crude oils can also include heteroatoms containing sulfur, nitrogen, nickel, vanadium and other elements in quantities that impact the refinery processing of the crude oil fractions. Light crude oils or condensates can contain sulfur in concentrations as low as 0.01 W %; in contrast, heavy crude oils can contain as much as 5-6 W %. Similarly, the nitrogen content of crude oils can range from 0.001-1.0 W %.

The nature of the crude oil governs, to a certain extent, the nature of the products that can be manufactured from it and their suitability for special applications. A naphthenic crude oil will be more suitable for the production of asphaltic bitumen, a paraffinic crude oil for wax. A naphthenic crude oil, and even more so an aromatic one, will yield lubricating oils with viscosities that are sensitive to temperature. However, with modern refining methods there is greater flexibility in the use of various crude oils to produce many desired type of products.

A crude oil assay is a traditional method of determining the nature of crude oils for benchmarking purposes. Crude oils are subjected to true boiling point (TBP) distillations and fractionations to provide different boiling point fractions. The crude oil distillations are carried out using the American Standard Testing Association (ASTM) Method D 2892. The common fractions and their nominal boiling points are given in Table 1.

TABLE 1

| Fraction | Boiling Point, ° C. |
|---|---|
| Methane | −161.5 |
| Ethane | −88.6 |
| Propane | −42.1 |
| Butanes | −6.0 |
| Light Naphtha | 36-90 |
| Mid Naphtha | 90-160 |
| Heavy Naphtha | 160-205 |
| Light gas Oil | 205-260 |
| Mid Gas Oil | 260-315 |
| Heavy gas Oil | 315-370 |
| Light Vacuum Gas Oil | 370-430 |
| Mid Vacuum Gas Oil | 430-480 |
| Heavy vacuum gas oil | 480-565 |
| Vacuum Residue | 565+ |

The yields, composition, physical and indicative properties of these crude oil fractions, where applicable, are then determined during the crude assay work-up calculations. Typical compositional and property information obtained from a crude oil assay is given in Table 2.

TABLE 2

| | | Property | |
|---|---|---|---|
| Property | Unit | Type | Fraction |
| Yield Weight and | W % | Yield | All |
| Volume % | | | |
| API Gravity | ° | Physical | All |
| Viscosity | ° | Physical | Fraction boiling >250° C. |
| Kinematic @ 38° C. | | | |
| Refractive | Unitless | Physical | Fraction boiling <400° C. |
| Index @ 20° C. | | | |
| Sulfur | W % | Composition | All |
| Mercaptan Sulfur, | W % | Composition | Fraction boiling <250° C. |
| W % | | | |
| Nickel | ppmw | Composition | Fraction boiling >400° C. |
| Nitrogen | ppmw | Composition | All |
| Flash Point, COC | ° C. | Indicative | All |
| Cloud Point | ° C. | Indicative | Fraction boiling >250° C. |
| Pour Point, | ° C. | Indicative | Fraction boiling >250° C. |
| (Upper) | | | |
| Freezing Point | ° C. | Indicative | Fraction boiling >250° C. |
| Microcarbon | W % | Indicative | Fraction boiling >300° C. |
| Residue | | | |
| Smoke Point, mm | mm | Indicative | Fraction boiling between |
| | | | 150-250 |
| Octane Number | Unitless | Indicative | Fraction boiling <250° C. |
| Cetane Index | Unitless | Indicative | Fraction boiling between |
| | | | 150-400 |
| Aniline Point | ° C. | Indicative | Fraction boiling <520° C. |

Due to the number of distillation cuts and the number of analyses involved, the crude oil assay work-up is both costly and time consuming.

In a typical refinery, crude oil is first fractionated in the atmospheric distillation column to separate sour gas and light hydrocarbons, including methane, ethane, propane, butanes and hydrogen sulfide, naphtha (36°-180° C.), kerosene (180°-240° C.), gas oil (240°-370° C.) and atmospheric residue (>370° C.). The atmospheric residue from the atmospheric distillation column is either used as fuel oil or sent to a vacuum distillation unit, depending on the configuration of the refinery. The principal products obtained from vacuum distillation are vacuum gas oil, comprising hydrocarbons boiling in the range 370°-520° C., and vacuum residue, comprising hydrocarbons boiling above 520° C. The crude assay data help refiners to understand the general composition of the crude oil fractions and properties so that the fractions can be processed most efficiently and effectively in an appropriate refining unit. Indicative properties are used to determine the engine/fuel performance or usability or flow characteristic or composition. A summary of the indicative properties and their determination methods with description are given below.

The cetane number of diesel fuel oil, determined by the ASTM D613 method, provides a measure of the ignition quality of diesel fuel; as determined in a standard single cylinder test engine; which measures ignition delay compared to primary reference fuels. The higher the cetane number; the easier the high-speed; direct-injection engine will start; and the less white smoking and diesel knock after start-up are. The cetane number of a diesel fuel oil is determined by comparing its combustion characteristics in a test engine with those for blends of reference fuels of known cetane number under standard operating conditions. This is accomplished using the bracketing hand wheel procedure which varies the compression ratio (hand wheel reading) for the sample and each of the two bracketing reference fuels to obtain a specific ignition delay, thus permitting interpolation of cetane number in terms of hand wheel reading.

The octane number, determined by the ASTM D2699 or D2700 methods, is a measure of a fuel's ability to prevent detonation in a spark ignition engine. Measured in a standard single-cylinder; variable-compression-ratio engine by comparison with primary reference fuels. Under mild conditions, the engine measures research octane number (RON), while under severe conditions, the engine measures motor octane number (MON). Where the law requires posting of octane numbers on dispensing pumps, the antiknock index (AKI) is used. This is the arithmetic average of RON and MON, (R+M)/2. It approximates the road octane number, which is a measure of how an average car responds to the fuel.

The cloud point, determined by the ASTM D2500 method, is the temperature at which a cloud of wax crystals appears when a lubricant or distillate fuel is cooled under standard conditions. Cloud point indicates the tendency of the material to plug filters or small orifices under cold weather conditions. The specimen is cooled at a specified rate and examined periodically. The temperature at which cloud is first observed at the bottom of the test jar is recorded as the cloud point. This test method covers only petroleum products and biodiesel fuels that are transparent in 40 mm thick layers, and with a cloud point below 49° C.

The pour point of petroleum products, determined by the ASTM D97 method, is an indicator of the ability of oil or distillate fuel to flow at cold operating temperatures. It is the lowest temperature at which the fluid will flow when cooled under prescribed conditions. After preliminary heating, the sample is cooled at a specified rate and examined at intervals of 3° C. for flow characteristics. The lowest temperature at which movement of the specimen is observed is recorded as the pour point.

The aniline point, determined by the ASTM D611 method, is the lowest temperature at which equal volumes of aniline and hydrocarbon fuel or lubricant base stock are completely miscible. A measure of the aromatic content of a hydrocarbon blend is used to predict the solvency of a base stock or the cetane number of a distillate fuel. Specified volumes of aniline and sample, or aniline and sample plus n-heptane, are placed in a tube and mixed mechanically. The mixture is heated at a controlled rate until the two phases become miscible. The mixture is then cooled at a controlled rate and the temperature at which two separate phases are again formed is recorded as the aniline point or mixed aniline point.

To determine these properties of gas oil or naphtha fractions conventionally, these fractions have to be distilled from the crude oil and then measured/identified using various analytical methods that are laborious, costly and time-consuming.

High pressure liquid chromatography or high-performance liquid chromatography (HPLC) is a technique that can separate a mixture of compounds into individual analytes or into a group of analytes, depending on the complexity of the sample. HPLC serves various analytical applications including identification, quantification and/or purification of an individual component or a group of components having similar properties.

HPLC operates in various modes, such as normal phase, reversed phase, ion exchange, gel permeation, and hydrophilic interaction, which are defined by the combination of stationary phases and mobile phases. For the present application, normal phase chromatography is used; where the stationary phase is polar and the mobile phase is non-polar. HPLC typically utilizes different types of stationary phases for different applications, a pump that moves the mobile phase(s) and analytes through the column, and a detector to provide a characteristic retention time for the analytes. The detector may also provide additional information related to the analytes. Analyte retention time varies depending on the strength of its interactions with the stationary phase, the ratio/composition of solvent(s) used, and the flow rate of the mobile phase.

Any new rapid, direct method to help better understand the crude oil composition and properties from the analysis of whole crude oil will save producers, marketers, refiners and/or other crude oil users substantial expense, effort and time. Therefore, a need exists for an improved system and method for determining the properties of crude oil fractions from different sources and classifying the crude oil fractions based on their boiling point characteristics and/or properties.

### SUMMARY OF THE INVENTION

The above objects and further advantages are provided by the present invention which broadly comprehends a system and a method for determining the indicative properties of a hydrocarbon sample. In accordance with the invention, indicative properties (i.e., cetane number, pour point, cloud point and aniline point of gas oil fraction and octane number of gasoline fraction in crude oils) are predicted by density and HPLC measurement of crude oils. The correlations also provide information about the gas oil properties without fractionation/distillation (crude oil assays) and will help producers, refiners, and marketers to benchmark the oil quality and, as a result, valuate the oils without performing the customary extensive and time-consuming crude oil assays.

### BRIEF DESCRIPTION OF THE DRAWINGS

Further advantages and features of the present invention will become apparent from the following detailed description of the invention when considered with reference to the accompanying drawings in which:

FIG. 1 is a graphic plot of typical HPLC data for a typical crude oil sample;

FIG. 2 is a block diagram of a method in which an embodiment of the invention is implemented;

FIG. 3 is a schematic block diagram of modules of an embodiment of the invention; and

FIG. 4 is a block diagram of a computer system in which an embodiment of the invention is implemented.

### DETAILED DESCRIPTION OF INVENTION

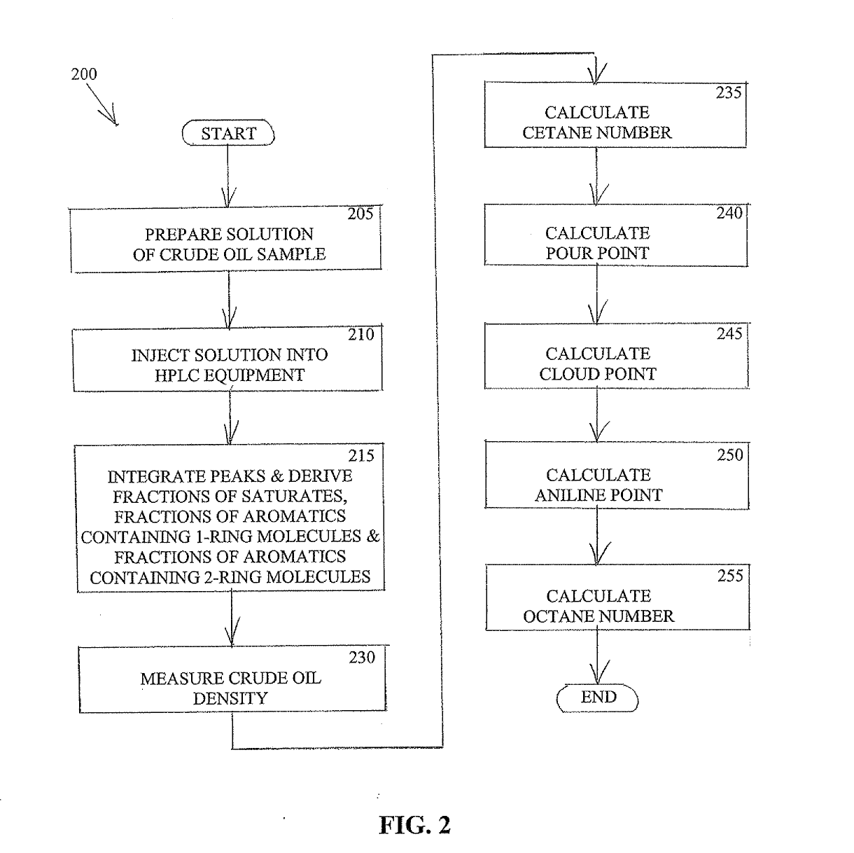

Crude oil samples were prepared and analyzed by HPLC according to the method 200 described below, and illustrated in FIG. 2.

In step 205, about 100 mg of crude oil sample is dissolved in 1 mL heptane. The solution is shaken thoroughly by a Vortex Mixer and allowed to stand for 10 minutes, after which it is filtered through a 0.45 μm Millipore filter in order to remove insoluble particles.

In step 210, the filtered sample is then directly injected into the HPLC equipment, consisting of a micro vacuum degasser, binary pump, column thermostat, auto-sampler and refractive index detector. The HPLC conditions maintained for this analysis are presented in the Table 3.

TABLE 3

| Flow rate; | 0.8 | mL/min |
|---|---|---|
| Detector: | Refractive index detector, | |
| | Optical unit temperature 30° C. | |
| Injection volume: | 10 | μL |
| Run time: | 40 | minutes |
| Forward flush | 11.4 | minute |
| Back flush | 11.4-30 | minute |
| Mobile phase: | n-heptane | |
| Column storage: | n-heptane | |
| Injector needle wash: | n-heptane | |
| Column: | LiChrospher NH₂ (250 × 4.6 mm) | |
| Column temperature: | 25° | C. |

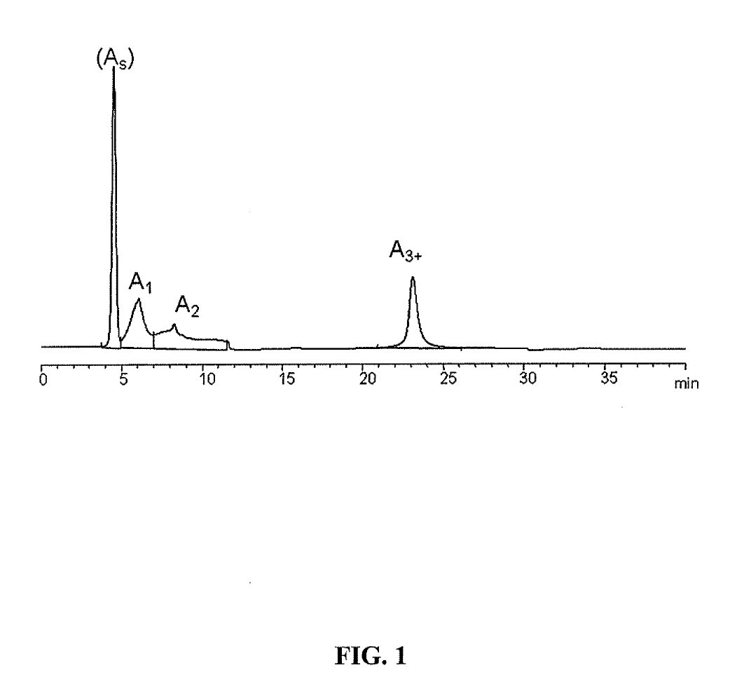

As the chromatogram of FIG. 1 shows, four peaks are obtained. The area under these peaks are: Aₛ, the peak area of saturates; A₁, the peak area of 1-aromatic rings; A₂, the peak area of 2-aromatic rings; and A₃₊, the peak area of 3+-aromatic rings.

In step 215, the peaks are manually integrated using Agilent ChemStation software, though any similar software may be used. The relevant percentage of each peak is reported as:

Fraction of saturates (SAT)=A_{S}/(A_{S}+A₁+A₂+A₃₊) (1);

Fraction of aromatics containing 1-ring molecules (AROM-1R)=A₁/(A_{S}+A₁+A₂+A₃₊) (2);

Fraction of aromatics containing 2-ring molecules (AROM-2R)=A₂/(A_{S}+A₁+A₂+A₃₊) (3);

Fraction of aromatics containing 3 or more aromatic rings=A₃₊/(A_{S}+A₁+A₂+A₃₊) (4).

The indicative properties (i.e., the cetane number, pour point, cloud point and aniline point of the gas oil fraction boiling in the range 180-370° C. and octane number for gasoline fraction boiling in the range 36-180° C.) of the crude oil can be predicted from the density of whole crude oil and the group type composition (saturates, 1-, 2-ring aromatics) of crude oil, as determined by HPLC. That is,

Indicative Property=f(density_{crude oil},SAT_{crude oil},AROM-1R_{crude oil},AROM-2R_{crude oil}) (5);

Equations (6) through (9) show, respectively, the cetane number, pour point, cloud point and aniline point of gas oils boiling in the range 180-370° C., and equation (10) shows the octane number of gasoline boiling in the range 36-180° C. that can be predicted from the density (which is determined in step 230) and 1- and/or 2-ring aromatics composition of crude oils. Thus, in step 235, the octane number is calculated as:

Cetane Number (CET)=K_{CET}+X1_{CET}*DEN+X2_{CET}*SAT+X3_{CET}*AROM-1R+X4_{CET}*AROM-2R (6);

In step 240, the pour point is calculated as:

Pour Point (PPT)=K_{PPT}+X1_{PPT}*DEN+X2_{PPT}*SAT+X3_{PPT}*AROM-1R+X4_{PPT}*AROM-2R (7)

In step 245, the cloud point is calculated as:

Cloud Point (CPT)=K_{CPT}+X1_{CPT}*DEN+X2_{CPT}*SAT+X3_{CPT}*AROM-1R+X4_{CPT}*AROM-2R (8)

In step 250, the aniline point is calculated as:

Aniline Point (AP)=K_{AP}+X1*DEN+X2*SAT+X3_{AP}*AROM-1R+X4_{AP}*AROM-2R (9)

In step 255, the octane number is calculated as:

Octane Number (ON)=K_{ON}+X1_{ON}*DEN+X2_{ON}*SAT+X3_{ON}*AROM-1R (10)

where:

DEN=density of the crude oil sample;

SAT=Fraction of saturates by HPLC;

AROM-1R=Fraction of aromatics containing 1-ring molecules by HPLC;

AROM-2R=Fraction of aromatics containing 2-ring molecules by HPLC; and

K_{CET}, X1_{CET}-X4_{CET}, K_{PPT}, X1_{PPT}-X4_{PPT}, K_{CPT}, X1_{CPT}-X4_{CPT}, K_{AP}, X1_{AP}-X4_{AP}, K_{ON}, X1_{ON}-X3_{ON} are constants that were developed using linear regression analysis of hydrocarbon data from HPLC, and which are given in Table 4.

TABLE 4

| | Cetane | Pour | Cloud | Aniline | Octane |
|---|---|---|---|---|---|
| Constants | Number | Point | Point | Point | Number |
| K | 272.6 | 355.3 | 265.2 | 234.3 | 273.4 |
| X1 | −220.8 | −425.5 | −317.9 | −163.9 | −230.8 |
| X2 | −79.3 | −46.7 | −25.0 | −43.8 | 36.2 |
| X3 | 5.9 | 22.4 | 5.8 | −13.4 | −138.9 |
| X4 | −3.3 | 60.4 | 37.0 | −38.6 | — |

The following example is provided to demonstrate an application of equations (6) through (10). A sample of Arabian medium crude with a 15° C./4° C. density of 0.8828 Kg/l was analyzed by HPLC, using the described method. The HPLC composition was determined to be: Saturates=27.2 W %; 1-Ring Aromatics=18.6 W %; 2-Ring Aromatics 28.5 W %; and 3+-Ring Aromatics=25.7 W %.

Applying equation (6) and the constants from Table 4,

$$\begin{matrix}Cetane Number ( CET ) = {K}_{CET} + X {1}_{CET} * DEN + X {2}_{CET} * SAT + \\ X {3}_{CET} * AROM \text{-} 1 R + X {4}_{CET} * AROM \text{-} 2 R \\ = ( 272.6 ) + ( - 220.8 ) ( 0.8828 ) + \\ ( - 79.3 ) ( 0.272 ) + ( 5.9 ) ( 0.186 ) + \\ ( - 3.3 ) ( 0.285 ) \\ = 56.3\end{matrix}$$

Applying equation (7) and the constants from Table 4,

$$\begin{matrix}Pour Point ( PPT ) = {K}_{PPT} + X {1}_{PPT} * DEN + X {2}_{PPT} * \\ SAT + X {3}_{PPT} * AROM \text{-} 1 R + \\ X {4}_{PPT} * AROM \text{-} 2 R \\ = ( 355.3 ) + ( - 425.5 ) ( 0.8828 ) + \\ ( - 46.7 ) ( 0.272 ) + ( 22.4 ) ( 0.186 ) + \\ ( 60.4 ) ( 0.285 ) \\ = - 12\end{matrix}$$

Applying equation (8) and the constants from Table 4,

$$\begin{matrix}Cloud Point ( CPT ) = {K}_{CPT} + X {1}_{CPT} * DEN + X {2}_{CPT} * SAT + \\ X {3}_{CPT} * AROM \text{-} 1 R + X {4}_{CPT} * AROM \text{-} 2 R \\ = ( 265.2 ) + ( - 317.9 ) ( 0.8828 ) + \\ ( - 25.0 ) ( 0.272 ) + ( 5.8 ) ( 0.186 ) + \\ ( 37.0 ) ( 0.285 ) \\ = - 11\end{matrix}$$

Applying equation (9) and the constants from Table 4,

$$\begin{matrix}Aniline Point ( AP ) = {K}_{AP} + X {1}_{AP} * DEN + X {2}_{AP} * SAT + \\ X {3}_{AP} * AROM \text{-} 1 R + X {4}_{AP} * AROM \text{-} 2 R \\ = ( 234.3 ) + ( - 163.9 ) ( 0.8828 ) + \\ ( - 43.8 ) ( 0.272 ) + ( - 13.4 ) ( 0.186 ) + \\ ( - 38.6 ) ( 0.285 ) \\ = 64\end{matrix}$$

Applying equation (10) and the constants from Table 4,

$$\begin{matrix}Octane Number ( ON ) = {K}_{ON} + X {1}_{ON} * DEN + X {2}_{ON} * \\ SAT + X {3}_{ON} * AROM \text{-} 1 R \\ = ( 273.4 ) + ( - 230.8 ) ( 0.8828 ) + \\ ( 36.2 ) ( 0.272 ) + ( - 138.9 ) ( 0.186 ) \\ = 54\end{matrix}$$

The method is applicable for naturally occurring hydrocarbons derived from crude oils, bitumens, heavy oils, shale oils and from refinery process units including hydrotreating, hydroprocessing, fluid catalytic cracking, coking, and visbreaking or coal liquefaction.

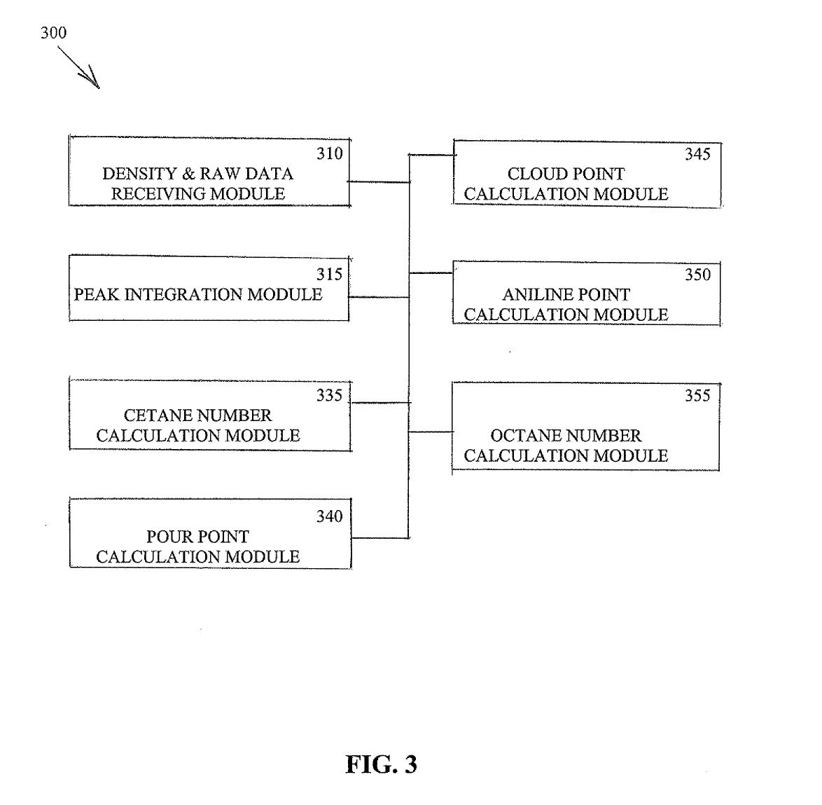

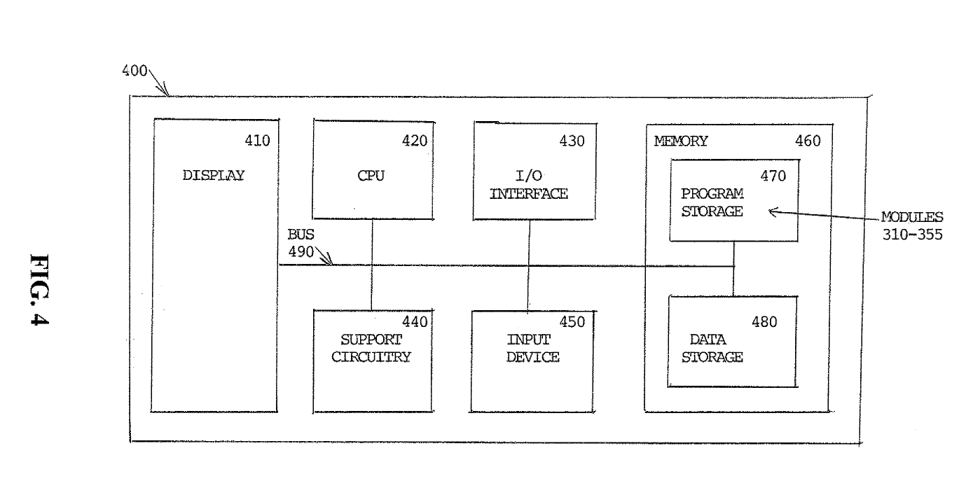

FIG. 3 illustrates a schematic block diagram of modules in accordance with an embodiment of the present invention, system 300. Density and raw data receiving module 310 receives the density of a sample of crude oil and high pressure liquid chromatography (HPLC) data derived from the crude oil. Peak integrating module 315 integrates the peaks derived from the HPLC and calculates the fraction of saturates, fraction of aromatics containing 1-ring molecules and fraction of aromatics containing 2-ring molecules.

Cetane number calculation module 335 derives the cetane number for the gas oil fraction as a function of the density of the sample, the fraction of saturates, the fraction of aromatics containing 1-ring molecules, and the fraction of aromatics containing 2-ring molecules.

Pour point calculation module 340 derives the pour point for the gas oil fraction as a function of the density of the sample, the fraction of saturates, the fraction of aromatics containing 1-ring molecules, and the fraction of aromatics containing 2-ring molecules.

Cloud point calculation module 345 derives the cloud point for the gas oil fraction as a function of the density of the sample, the fraction of saturates, the fraction of aromatics containing 1-ring molecules, and the fraction of aromatics containing 2-ring molecules.

Aniline point calculation module 350 derives the aniline point for the gas oil fraction as a function of the density of the sample, the fraction of saturates, the fraction of aromatics containing 1-ring molecules, and the fraction of aromatics containing 2-ring molecules.

Octane number calculation module 355 derives the octane number for the gasoline fraction as a function of the density of the sample, the fraction of saturates, the fraction of aromatics containing 1-ring molecules, and the fraction of aromatics containing 2-ring molecules.

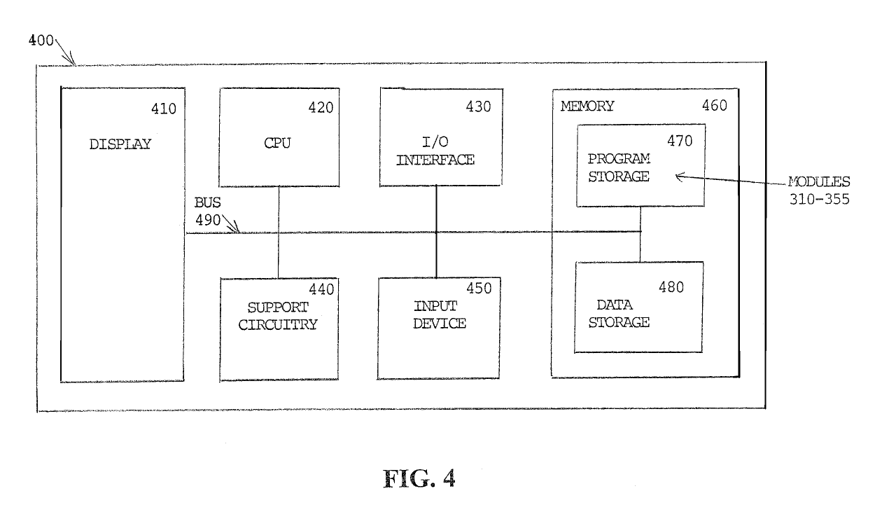

FIG. 4 shows an exemplary block diagram of a computer system 400 in which the partial discharge classification system of the present invention can be implemented. Computer system 400 includes a processor 420, such as a central processing unit, an input/output interface 430 and support circuitry 440. In certain embodiments, where the computer system 400 requires a direct human interface, a display 410 and an input device 450 such as a keyboard, mouse or pointer are also provided. The display 410, input device 450, processor 420, and support circuitry 440 are shown connected to a bus 490 which also connects to a memory 460. Memory 460 includes program storage memory 470 and data storage memory 480. Note that while computer system 400 is depicted with direct human interface components display 410 and input device 450, programming of modules and exportation of data can alternatively be accomplished over the input/output interface 430, for instance, where the computer system 400 is connected to a network and the programming and display operations occur on another associated computer, or via a detachable input device as is known with respect to interfacing programmable logic controllers.

Program storage memory 470 and data storage memory 480 can each comprise volatile (RAM) and non-volatile (ROM) memory units and can also comprise hard disk and backup storage capacity, and both program storage memory 470 and data storage memory 480 can be embodied in a single memory device or separated in plural memory devices. Program storage memory 470 stores software program modules and associated data, and in particular stores a density and raw data receiving module 310, peak integrating module 315, cetane number calculation module 330, pour point calculation module 340, cloud point calculation module 345, aniline point calculation module 350, and octane number calculation module 355. Data storage memory 480 stores results and other data generated by the one or more modules of the present invention.

It is to be appreciated that the computer system 400 can be any computer such as a personal computer, minicomputer, workstation, mainframe, a dedicated controller such as a programmable logic controller, or a combination thereof. While the computer system 400 is shown, for illustration purposes, as a single computer unit, the system can comprise a group of computers which can be scaled depending on the processing load and database size.

Computer system 400 preferably supports an operating system, for example stored in program storage memory 470 and executed by the processor 420 from volatile memory. According to an embodiment of the invention, the operating system contains instructions for interfacing computer system 400 to the Internet and/or to private networks.

One of ordinary skill in the art will also comprehend that an embodiment of the partial discharge classification method of the present invention can be provided in the form of a computer program product.

The system and method of the present invention have been described above and with reference to the attached figure; however, modifications will be apparent to those of ordinary skill in the art and the scope of protection for the invention is to be defined by the claims that follow.

We claim:

1. A system for determining indicative properties of gasoline and gas oil fractions of crude oil, based upon high pressure liquid chromatography (HPLC) data derived from the corresponding crude oil and the density of the sample, the system comprising:
a non-volatile memory device that stores calculation modules and data;
a processor coupled to the memory;
a first calculation module that calculates the saturates and aromatic composition of the gas oil fraction from the HPLC data;
a second calculation module that derives the cetane number for the gas oil fraction as a function of the saturates and aromatic composition and the density of the sample;
a third calculation module that derives the pour point for the gas oil fraction as a function of the saturates and aromatic composition and the density of the sample;
a fourth calculation module that derives the cloud point for the gas oil fraction as a function of the saturates and aromatic composition and the density of the sample;
a fifth calculation module that derives the aniline point for the gas oil fraction as a function of the saturates and aromatic composition and the density of the sample; and
a sixth calculation module that derives the octane number for the gasoline fraction as a function of the saturates and aromatic composition and the density of the sample.

2. The system of claim 1, wherein the gas oil boils in the nominal range 180° C.-370° C.

3. The system of claim 1, wherein the gasoline boils in the nominal range 36° C.-180° C.

4. A method for operating a computer to determine indicative properties of gasoline and gas oil fractions of crude oil based upon a sample of the crude oil taken from an oil well, stabilizer, extractor, or distillation tower, the method comprising:
obtaining the density of the crude oil sample;
preparing the crude oil sample for high pressure liquid chromatography (HPLC) analysis;
obtaining chromatogram data for the crude oil sample by HPLC analysis;
entering into the computer HPLC peak area data obtained by HPLC analysis of the crude oil sample;
calculating saturates and aromatics content of the gas oil fraction from the HPLC peak area data;
calculating the cetane number for the gas oil fraction as a function of the density and saturates and aromatics content of the sample;
calculating the pour point for the gas oil fraction as a function of the density and saturates and aromatics content of the sample;
calculating the cloud point for the gas oil fraction as a function of the density and saturates and aromatics content of the sample;
calculating the aniline point for the gas oil fraction as a function of the density and saturates and aromatics content of the sample; and
calculating the octane number for the gasoline fraction as a function of the density and saturates and aromatics content of the sample.

5. A computer program product to determine indicative properties of gasoline and gas oil fractions of crude oil based upon a sample of the crude oil taken from an oil well, stabilizer, extractor, or distillation tower, comprising a non-transitory computer readable medium having computer readable program code embodied therein that, when executed by a processor, causes the processor to:
accept the value of the density of the crude oil sample;
accept or calculate the fraction of saturates, fraction of aromatics containing 1-ring molecules, and the fraction of aromatics containing 2-ring molecules;
calculate the cetane number for the gas oil fraction as a function of the density, the fraction of the saturates, the fraction of aromatics containing 1-ring molecules, and the fraction of aromatics containing 2-ring molecules;
calculate the pour point for the gas oil fraction as a function of the density, the fraction of the saturates, the fraction of aromatics containing 1-ring molecules, and the fraction of aromatics containing 2-ring molecules;
calculate the cloud point for the gas oil fraction as a function of the density, the fraction of the saturates, the fraction of aromatics containing 1-ring molecules, and the fraction of aromatics containing 2-ring molecules;
calculate the aniline point for the gas oil fraction as a function of the density, the fraction of the saturates, the fraction of aromatics containing 1-ring molecules, and the fraction of aromatics containing 2-ring molecules;
calculate the octane number for the gasoline fraction as a function of density, the fraction of the saturates, the fraction of aromatics containing 1-ring molecules, and the fraction of aromatics containing 2-ring molecules; and
display the calculated results and/or store the calculated results into memory.

6. The computer program product of claim 5, further comprising a non-transitory computer readable medium having computer readable program code embodied therein that, when executed by a processor, causes the processor to:
accept data obtained by high pressure liquid chromatography (HPLC) analysis of the crude oil sample; and
integrate the HPLC peaks and calculate the fraction of saturates, fraction of aromatics containing 1-ring molecules, and the fraction of aromatics containing 2-ring molecules.
